(19) United States
(12) Patent Application Publication
(10) Pub. No.: US 2019/0116893 A1
(43) Pub. Date: Apr. 25, 2019

(54) MULTI-FUNCTION BREASTFEEDING AND PUMPING GARMENT

(71) Applicant: Rachel Kathryn Blacker, Las Vegas, NV (US)

(72) Inventor: Rachel Kathryn Blacker, Las Vegas, NV (US)

(21) Appl. No.: 15/790,340

(22) Filed: Oct. 23, 2017

(51) Int. Cl. A41D 1/20; A61M 1/06

(52) U.S. Cl. CPC A41D 1/205; A61M 1/062

(57) ABSTRACT

An apparatus and system for facilitating breast milk pumping and breastfeeding in a discreet and comfortable manner. The apparatus including a bandeau provided within a covering shirt. The bandeau can include a pair of apertures corresponding in location to a wearer's nipples which provide access through the bandeau to the wearer's nipples for pumping purposes. Further, the bandeau and covering shirt being formed of a resilient material for exposing one or both breasts, or alternatively uncovering only the bandeau.

### PRIORITY CLAIM

Priority is claimed to co-pending U.S. patent application Ser. No. 14/791,938 filed Jul. 6, 2015, which is hereby incorporated herein by reference in its entirety.

### COPYRIGHT STATEMENT

A portion of the disclosure of this patent document contains material which is subject to (copyright or mask work) protection. The (copyright or mask work) owner has no objection to the facsimile reproduction by anyone of the patent document or the patent disclosure, as it appears in the Patent and Trademark Office patent file or records, but otherwise reserves all (copyright or mask work) rights whatsoever.

### BACKGROUND

### Field of the Invention

The present invention relates generally to consumer maternity products. More particularly, the present invention relates to a shirt and bandeau combination for use by mothers who desire to nurse their children or pump breast milk in a modest yet comfortable and accessible manner.

### Related Art

It will be appreciated by those skilled in the art that babies are often in need of their mother's milk, and that the feeding schedule of babies does not always necessarily coincide with the external scheduling demands of new mothers. As such, babies will often need to breastfeed in public, or in otherwise less than private situations. Additionally, the mother will not necessarily always be with their baby when the lactation begins or the breastmilk otherwise needs to be extracted. As such, mothers often utilize breast pumps in order to extract milk and preserve it for a later feeding of the baby using, for example, a bottle.

Various nursing bras presently available to nursing mothers typically include large panels which can be unclipped allowing the panels to be pulled away, thus exposing the breast and allowing access of the child or a breast pump cone to the exposed breast. Problems arise because outer clothing items, i.e. shirts, blouses, etc., which are suitable for everyday use in public, do not typically allow for easy access to the breasts without substantial inconvenience, often requiring being pulled up from the bottom so as to expose substantial portions of the stomach and shoulders in order to properly gain access to a desired breast, wherein such significant exposure is often not desired, such as in public locations. Some products also involve using large blanket type covers which can cover the mother's torso such that the mother can sufficiently undress under the cover. One particular problem with such covers is that breastfeeding babies often dislike being covered while awake and feeding and can push the cover away, which exposes the undressed mother's torso, almost completely. Such covers also still require a significant amount of undressing under the cover.

It will be further appreciated that providing more secure and convenient comfort to nursing mothers is a continuing endeavor. The present invention overcomes many of the deficiencies present in the products presently available. Some such advantages will be discussed in more detail below.

### SUMMARY OF THE INVENTION

Contemplated herein is a multi-function breastfeeding garment, the garment including an outer shirt, the outer shirt having a pair of shoulder portions configured to extend over a user's shoulders. Within the outer shirt an inner bandeau is provided being generally cylindrical in shape and having an annular wall structure, an upper edge, and a lower edge, the upper edge being parallel to the lower edge about an entire circumference of the bandeau, the bandeau being configured to wrap around the user's upper torso, the upper edge of the bandeau being configured to rest completely below the user's armpits, and the lower edge of the bandeau configured to rest above the user's stomach

A pair of apertures can then be formed in a front portion of the annular wall structure, the pair of apertures extending from an exterior surface to an inner surface, the pair of apertures being further configured to allow access to the user's nipples without removal of the bandeau.

In order to provide increased breast support, two pairs of opposing attachment mechanisms provided about the upper edge of the bandeau which correspond with attachment of a pair of support straps being configured to connect to and extend between respective attachment mechanisms and extend over the user's shoulders.

In some embodiments a seam can be provided being configured to couple the bandeau to the outer shirt around a rear or spinal portion.

In some alternative embodiments, each aperture can be provided as a slit which extends horizontally. In some such embodiments each aperture can be further configured to rest about an under side of each of the user's respective breasts when worn.

In yet additional embodiments, each support strap can also include a tension adjustment mechanism being provided along a respective length of each support strap, wherein in some such embodiments the tension adjustment mechanism can also be provided about a front portion of each support strap.

In some alternative embodiments, the opposing attachment mechanisms can include loops configured to releasably attach to corresponding hooks provided about the ends of the support straps.

In yet additional embodiments, an upper seam of the bandeau can be provided as an elastic seam, the elastic seam being configured to resiliently stretch over and expose the user's breast without plastic deformation.

In yet additional embodiments, the outer shirt can also be provided with an elastic seam through a shoulder portion or along a neck portion, wherein the elastic seam can be configured to resiliently stretch over and expose the front portion of the bandeau without plastic deformation.

In some alternative embodiments, a strap guide can be provided on an interior surface of each of the shoulder portions, wherein each support strap can then be configured to extend through a respective strap guide.

In yet additional embodiments contemplated herein, the seam coupling the bandeau to the outer shirt can be located along a spinal portion in a rear panel of both the bandeau and the outer shirt.

In another aspect of the present invention, various methods of providing and using a multi-function breastfeeding garment are contemplated herein wherein each of the components enumerated above are provided in varying combinations or used in varying manners as illustrated in the various figures.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 illustrates a perspective view of a front portion of a multi-function breast feeding and pumping garment in accordance with various aspects of the present invention;

FIG. 2 illustrates a front view of a multi-function breast feeding and pumping garment in accordance with the embodiment shown in FIG. 1;

FIG. 3 illustrates a rear or spinal view of a multi-function breast feeding and pumping garment in accordance with the embodiment shown in FIG. 1;

FIG. 4A illustrates a front view of the function of the multi-function breast feeding and pumping garment respectively without a strap support assembly illustrating a method of use in accordance with the embodiment shown in FIG. 1;

FIG. 4B illustrates a front view of the function of the multi-function breast feeding and pumping garment respectively with a strap support assembly illustrating another method of use in accordance with the embodiment shown in FIG. 1;

FIG. 5 illustrates a front view of another potential use of the multi-function breast feeding and pumping garment in accordance with the embodiment shown in FIG. 1;

FIG. 6 illustrates a front view of yet another potential use of the multi-function breast feeding and pumping garment in accordance with the embodiment shown in FIG. 1;

FIG. 7 illustrates a front perspective view of a front portion of the inner bandeau assembly for use in the multi-function breast feeding and pumping garment of FIG. 1;

FIG. 8 7 illustrates a rear perspective view of a front portion of the inner bandeau assembly of FIG. 7 for use in the multi-function breast feeding and pumping garment of FIG. 1;

FIG. 9A illustrates a cross-sectional side view of the multi-function breast feeding and pumping garment of FIG. 1; and

FIG. 9B illustrates an enlarged cross-sectional view of an indicated area encompassed by the dotted lines of FIG. 9A.

Reference will now be made to the exemplary embodiments illustrated, and specific language will be used herein to describe the same. It will nevertheless be understood that no limitation of the scope of the invention is thereby intended though the exemplary embodiments discussed, but the examples are for purposes of illustration of the inventive concepts.

### DETAILED DESCRIPTION OF EXAMPLE EMBODIMENT(S)

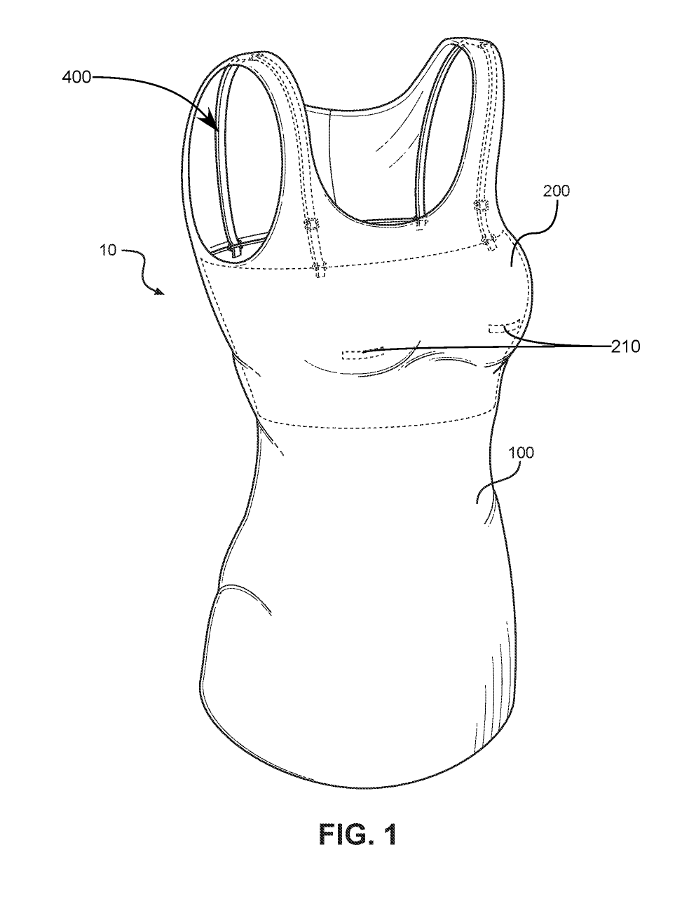

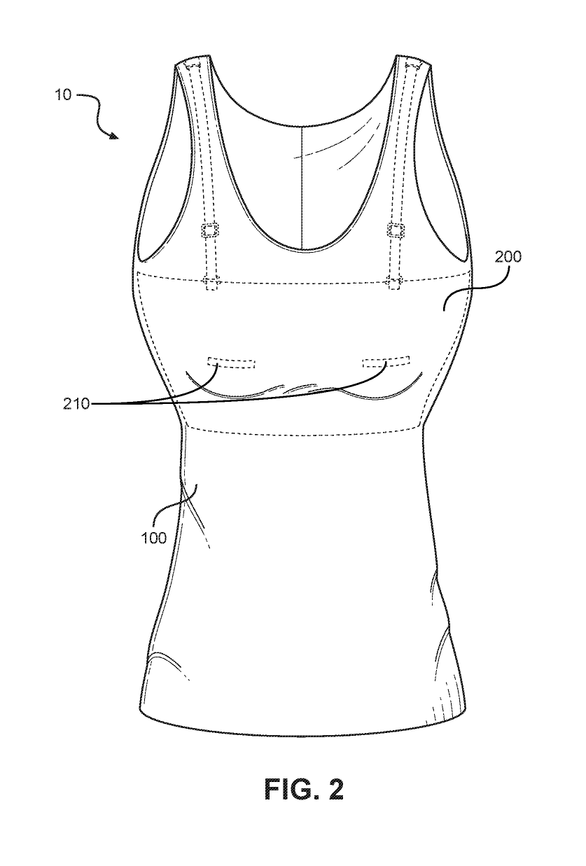

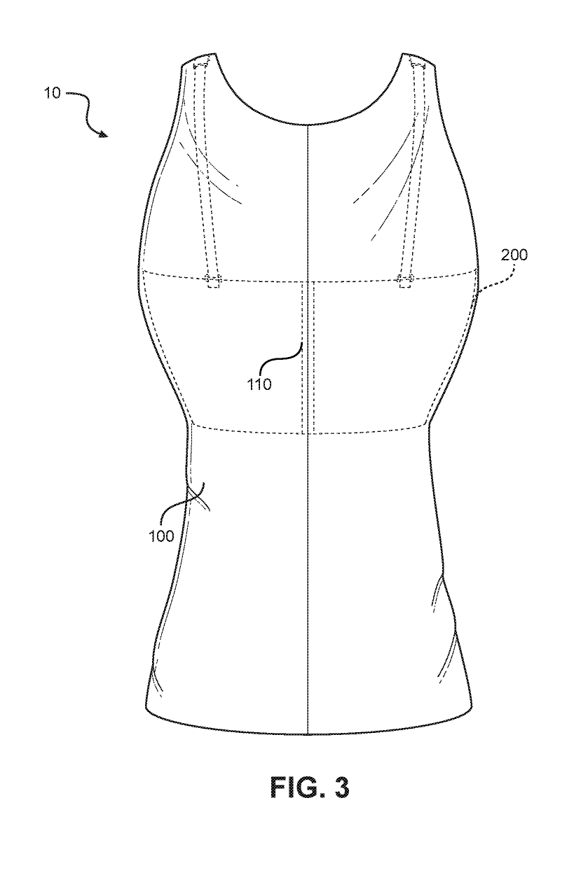

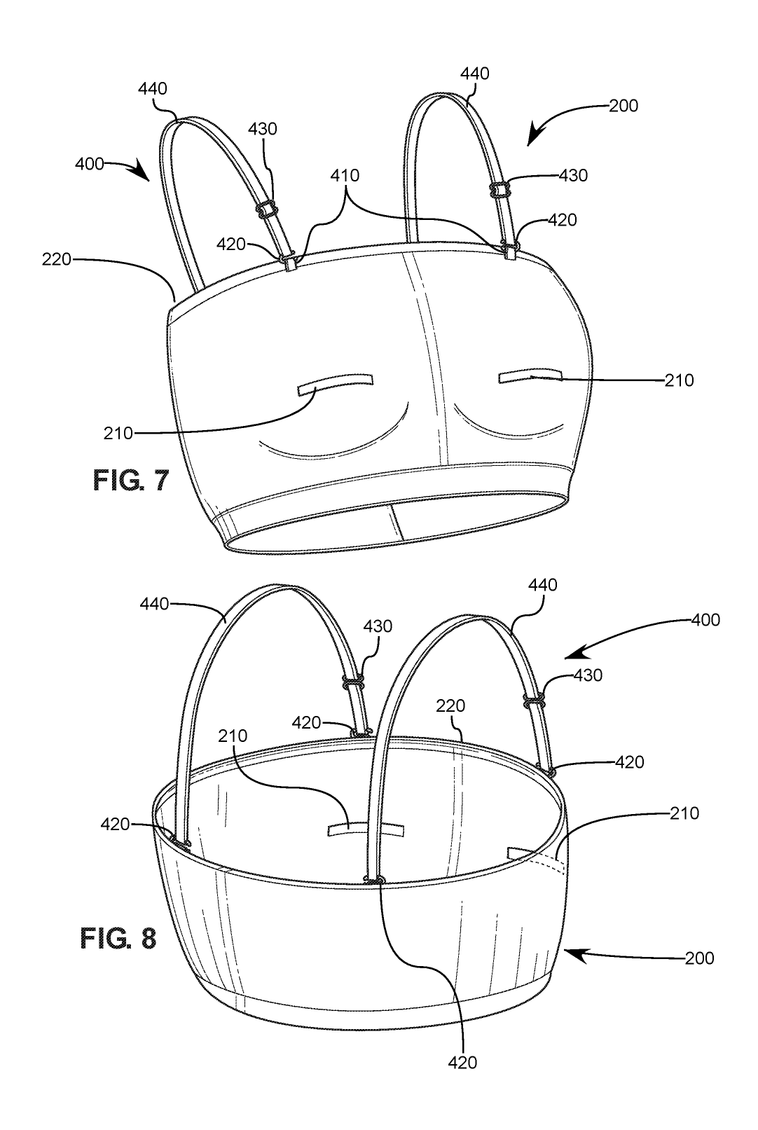

FIGS. 1-9, illustrate an exemplary embodiment of a multi-function breastfeeding garment 10 which overcomes various deficiencies of nursing garments or covers as existing in the prior art. The Garment is intended to be worn by a user, wherein the user is a nursing mother who either breastfeeds directly, pumps breast milk for bottle feeding, or both.

The multi-function breastfeeding garment 10 is intended to be suitable for wearing in public, be fashionable, and not readily apparent that the multi-function breastfeeding garment 10 is a piece of functional clothing.

As such the multi-function breastfeeding garment 10 includes two primary components, the first of which is an exterior shirt 100, the second of which is a bandeau 200 located and worn inside of the exterior shirt 100.

The bandeau 200 can be provided with a pair of apertures 210 which extend through the bandeau 200. The apertures 210 are located such that they provide access through the bandeau 200 to the user's nipples without requiring exposing any additional area of the user's breast.

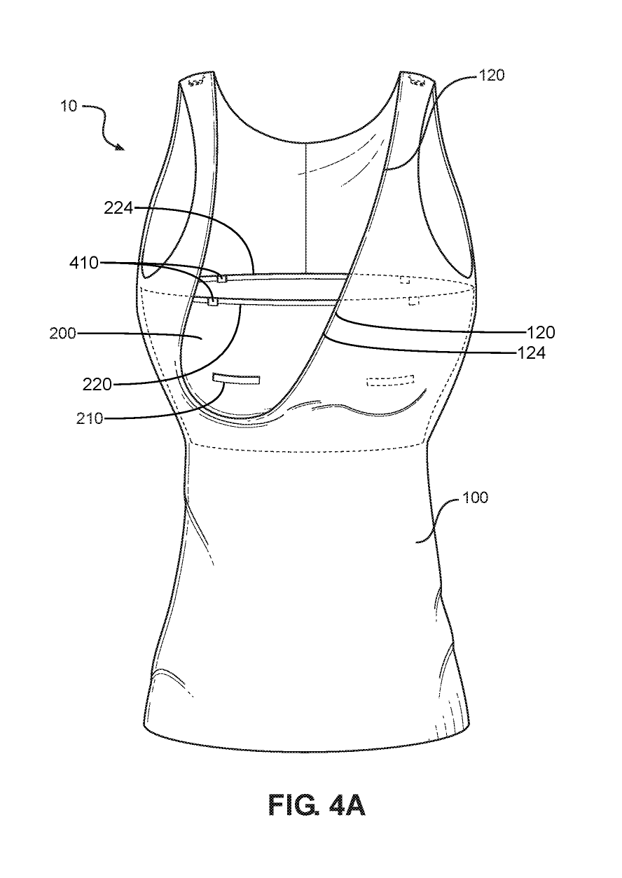

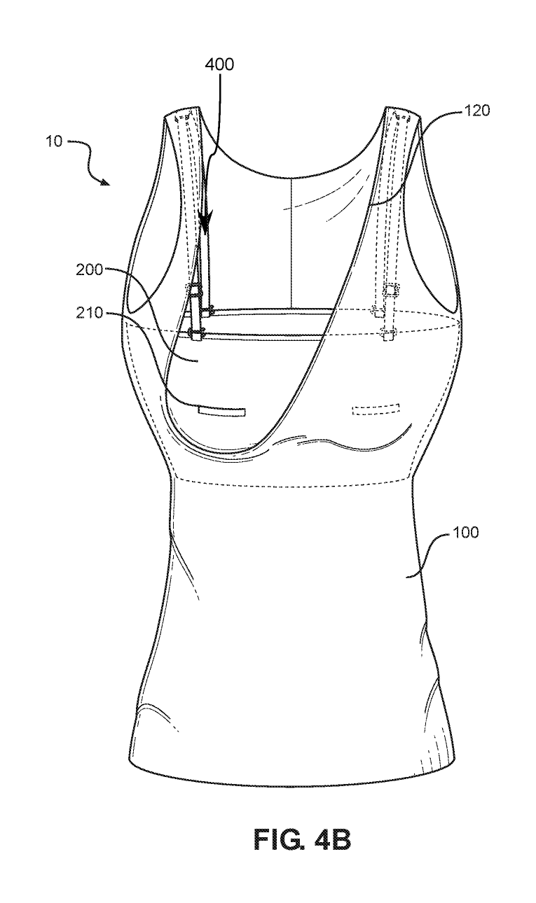

The exterior shirt 100 can be provided with an elastic seam 120 around a neck opening along a shoulder portion 124 as well as at the arms apertures such that the exterior shirt 100 can be pulled down to expose the bandeau 200 without complete or even partial removal of the exterior shirt. This pulled away configuration is illustrated in FIGS. 4-5, wherein the exterior shirt 100 is pulled down to access one or both of the user's nipples through the bandeau 200 for insertion of a pump or pumping accessories 300.

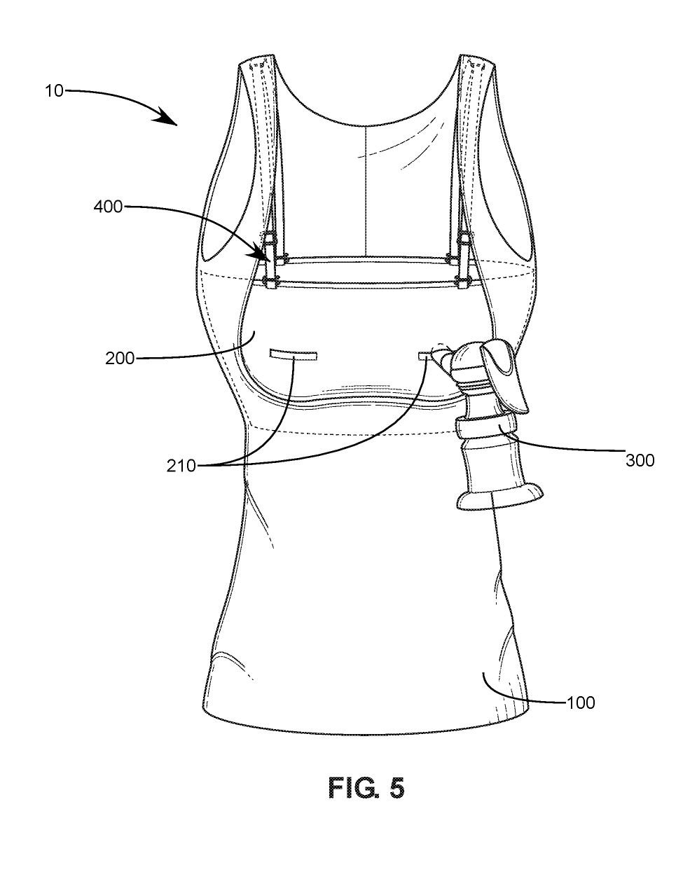

The bandeau can also be provided with an elastic seam 220 at an upper portion along an upper edge 224 such that it can also be pulled down to expose one or both of the user's breasts as seen in FIG. 6. This configuration can be useful for direct breastfeeding of a child or in instances wherein a user's breast pump, as illustrated by 300 in FIG. 5, is too difficult or uncomfortable to use through a desired aperture 210.

In other alternative instances, the exterior shirt 100 can be pulled down to fully expose the front portion of the bandeau 200, wherein the bandeau can be pulled down to selectively expose one breast to directly breastfeed a child, while the nipple of the other breast is accessed through the bandeau 200 to utilize a breast pump on the other breast.

In some embodiments the bandeau 200 and the exterior shirt 100 can be permanently affixed to one another via a seam 110 being provided about a rear portion of the bandeau and the outer shirt, i.e. about spinal portion of the user when worn. One or more such seams can be provided connecting the bandeau 200 to the exterior shirt 100, wherein in the case of multiple seams, they can be provided at different spacing or locations about an outer perimeter of the exterior shirt 100 and the bandeau 200. It has been recognized that a single seam provided from a top portion to a bottom portion along a spinal area has been particularly advantageous. It has further been appreciated that an expandable stitching method allows for the stretching of the material so as to better conform to variations in individual user body contours and dimensions. Further, flat seams also provide increased comfort when sitting and applying pressure, such as when sitting in a chair having back support, such that the seam does not provide an irritation point to the user. However, this seam can be provided along a flank portion at varying intervals as well, insomuch that the placement of the seams does not constrict the exterior shirt's and bandeau's ability to be pulled down in a front portion for accessing one or both breasts.

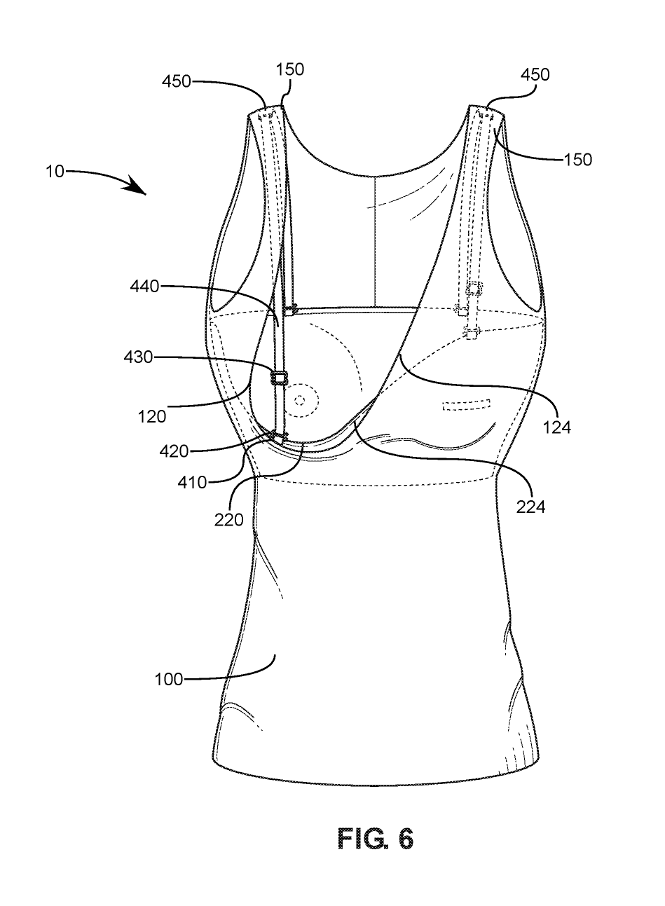

FIGS. 7-9 illustrate the bandeau 200 in more detail. In particular, FIGS. 7-9 illustrate various views of the bandeau 200 being separated from the outer shirt 100.

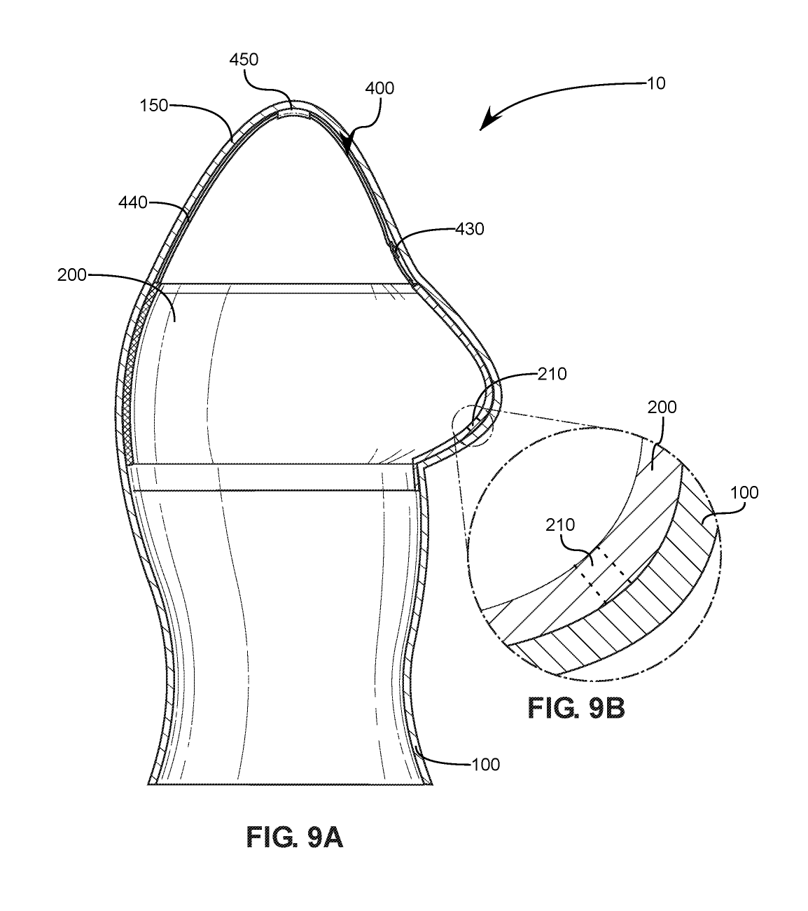

Additionally, as illustrated in the various figures shown herein, the apertures 210 can be provided as a pair of horizontal slits. It will be appreciated that the aperture locations can be provided in varying locations so as to be customizable to the user's individual nipple location. Alternatively, the apertures 210 can be provided in virtually any configuration, or any number of additional geometric patterns, “X” shapes, or any number of other conceivable configurations. In particular, slits can be advantageous because reinforcement stitching, similar to a button hole, can be provided so as to reinforce the aperture 210 and prevent tearing of the material forming the bandeau 200. The horizontal aperture orientation, as shown herein, can be of particular advantage because the apertures 210 can be located at a point below the curve of the breast. By providing the apertures 210 at a point below or underneath the natural curve of the breast can reduce instances of printing, i.e. viewing the contours of the aperture through the outer shirt when pressed or pulled tight through wearing or movement of the user/wearer. As such, location of the apertures underneath the breast below of most forward point of the breast will cause the apertures will be less visible as outer point of the breast will only show or cause printing of the solid or unbroken portions of the bandeau while retaining the apertures in a lower portion where the outer shirt and the bandeau will be less likely to be pressed together or pulled tight. In particular, FIGS. 9A-B illustrate the location of the apertures 210 below of farthest forward point of the user's breast, or natural cup of the outer shirt 100 or the bandeau 200 itself.

In yet additional embodiments, and as shown in FIGS. 1-9 the multi-function breastfeeding garment 10 as contemplated herein can further include strap support assembly 400. In order to facilitate connection of the strap support assembly two pairs of opposing attachment mechanisms 410 can be provided about the upper edge 220 of the bandeau 200. It will be appreciated that the opposing attachment mechanisms 410 can be provided as buttons, hooks, hook and loop fastener, stitching, etc. As illustrated herein the opposing attachment mechanisms are shown as a looped portion of fabric or material configured to receive a releasable hook 420 provided at an end portion of a pair of support straps 440 configured to connect to and extend between respective attachment mechanisms and extend over the user's shoulders. It will be appreciated that the support straps 440 can be provided as an elastic material which can be configured to stretch, while simultaneously providing sufficient support such that each support strap 440 can be pulled downward with the bandeau 200 as shown in FIG. 6.

However, the straps can also be configured to release easily prior to stretching downward when the user wishes to expose an entire breast. As such, it will also be appreciated that the support straps 440 can be configured to instead be released by unhooking the releasable hook 420 from a corresponding front attachment mechanism 410 so as to remove a potentially conflicting support strap 440.

Additionally, as some users may require more support, while other users require less. the support straps 440 can be configured to release from both the front and rear attachment mechanisms 410 so as to be fully removable for users who lack the need for the additional support provided thereby.

Additionally, the support straps 440 can be provided with a tension adjustment mechanism 430 which can be provided as a sliding buckle which can adjust the tension or the length of a corresponding support strap. It will be appreciated that these tension adjustment mechanisms 430 can be provided on a front portion of the support straps such that the tension can be easily adjusted without removal of the outer shirt and bandeau.

In yet additional embodiments, a strap guide 450 can be provided on an interior surface of each of the shoulder portions 150 of the outer shirt 100, wherein each support strap 440 is configured to extend through a respective strap guide 450. In this manner the support straps 440 can be effectively concealed behind the shoulder portions 150 and will thus reduce showing of the support straps 440 when the user is wearing the multi-function breastfeeding garment 10 and shifting or otherwise moving.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Further, discussion with regard to any of the specific features is intended to be for illustrative purposes, with the understanding that any feature discussed herein can be used in combination with any number of other features in any combination. Accordingly, it is not intended that the invention be limited, except as by the claims set forth below.

1. A multi-function breastfeeding garment, the garment comprising:
an outer shirt, the outer shirt having a pair of shoulder portions configured to extend over a user's shoulders;
an inner bandeau being generally cylindrical in shape and having an annular wall structure, an upper edge, and a lower edge, the upper edge being parallel to the lower edge about an entire circumference of the bandeau, the bandeau being configured to wrap around the user's upper torso, the upper edge of the bandeau being configured to rest completely below the user's armpits, and the lower edge of the bandeau configured to rest above the user's stomach, the bandeau further comprising:
a pair of apertures being formed in a front portion of the annular wall structure, the pair of apertures extending from an exterior surface to an inner surface, the pair of apertures being further configured to allow access to the user's nipples without removal of the bandeau; and
two pairs of opposing attachment mechanisms provided about the upper edge of the bandeau;
a pair of support straps configured to connect to and extend between respective attachment mechanisms and extend over the user's shoulders; and
a seam coupling the bandeau to the outer shirt.

2. The garment of claim 1, wherein each aperture is provided as a slit which extends horizontally.

3. The garment of claim 2, wherein each aperture is configured to rest about an under side of each of the user's respective breasts when worn.

4. The garment of claim 1, wherein each support strap further comprises a tension adjustment mechanism being provided along a respective length of each support strap.

5. The garment of claim 4, wherein the tension adjustment mechanism is provided about a front portion of each support strap.

6. The garment of claim 1, wherein the opposing attachment mechanisms include loops configured to releasably attach to corresponding hooks provided about the ends of the support straps.

7. The garment of claim 1, wherein an upper seam of the bandeau is provided as an elastic seam, the elastic seam being configured to resiliently stretch over and expose the user's breast without plastic deformation.

8. The garment of claim 7, wherein the outer shirt further comprises an elastic seam through a shoulder portion, the elastic seam being configured to resiliently stretch over and expose the front portion of the bandeau without plastic deformation.

9. The garment of claim 1, wherein the outer shirt further comprises an elastic seam through a shoulder portion, the elastic seam being configured to resiliently stretch over and expose the front portion of the bandeau without plastic deformation.

10. The garment of claim 1, further comprising:
a strap guide provided on an interior surface of each of the shoulder portions, wherein each support strap is configured to extend through a respective strap guide.

11. The garment of claim 1, wherein the seam coupling the bandeau to the outer shirt is located along a spinal portion in a rear panel of both the bandeau and the outer shirt.

12. A method of providing a multi-function breastfeeding garment, the method including:
providing an outer shirt;
providing an inner bandeau being generally cylindrical in shape and having an annular wall structure, an upper edge, and a lower edge, the upper edge being parallel to the lower edge about an entire circumference of the bandeau, the bandeau being configured to wrap around a user's upper torso, the upper edge of the bandeau being configured to rest completely below the user's armpits, and the lower edge of the bandeau configured to rest above the user's stomach;
providing two pairs of opposing attachment mechanisms about the upper edge of the bandeau;
providing a pair of support straps configured to connect to and extend between respective attachment mechanisms and extend over the user's shoulders;
providing a pair of apertures through a front portion of the annular wall structure, the pair of apertures extending from an exterior surface to an inner surface of the bandeau, the pair of apertures being further configured to allow access to the user's nipples without removal of the bandeau; and
affixing the bandeau to the outer shirt utilizing a sewn seam.

13. The method of claim 12, wherein each aperture is provided as a slit which extends horizontally.

14. The method of claim 13, wherein each aperture is configured to rest about an under side of each of the user's respective breasts when worn.

15. The method of claim 12, wherein each support strap further comprises a tension adjustment mechanism being provided along a respective length of each support strap.

16. The method of claim 15, wherein the tension adjustment mechanism is provided about a front portion of each support strap.

17. The method of claim 12, wherein the opposing attachment mechanisms include loops configured to releasably attach to corresponding hooks provided about the ends of the support straps.

18. The method of claim 12, further comprising:
providing an elastic seam along the upper edge of the, the elastic seam being configured to resiliently stretch over and expose the user's breast without plastic deformation; and
providing a second elastic seam through a shoulder portion of the outer shirt, the second elastic seam being configured to resiliently stretch over and expose the front portion of the bandeau without plastic deformation.

19. The method of claim 12, wherein the seam coupling the bandeau to the outer shirt is located along a spinal portion in a rear panel of both the bandeau and the outer shirt.

20. The method of claim 12, further comprising:
providing a strap guide on an interior surface of each of the shoulder portions, wherein each support strap is configured to extend through a respective strap guide.
